(19) United States
(12) Patent Application Publication
(10) Pub. No.: US 2018/0105840 A1
(43) Pub. Date: Apr. 19, 2018

(54) PROCESS FOR INDUSTRIAL PRODUCTION OF CHIRAL-1,1-DIFLUORO-2-PROPANOL

(71) Applicants: PUBLIC UNIVERSITY CORPORATION TOYAMA PREFECTURAL UNIVERSITY, Imizu-shi, Toyama (JP); CENTRAL GLASS COMPANY, LIMITED, Ube-shi, Yamaguchi (JP)

(72) Inventors: Yasuhisa ASANO, Toyama (JP); Kimiyasu ISOBE, Toyama (JP); Ryoko UEDA, Toyama (JP); Tetsuro NISHII, Saitama (JP); Akihiro ISHII, Saitama (JP)

(73) Assignees: PUBLIC UNIVERSITY CORPORATION TOYAMA PREFECTURAL UNIVERSITY, Imizu-shi, Toyama (JP); CENTRAL GLASS COMPANY, LIMITED, Ube-shi, Yamaguchi (JP)

(21) Appl. No.: 15/554,999

(22) PCT Filed: Mar. 2, 2016
(86) PCT No.: PCT/JP2016/056325

§ 371 Date: Aug. 31, 2017

(30) Foreign Application Priority Data
Mar. 3, 2015 (JP) 2015-041696

(51) Int. Cl. C12P 7/04; C12N 1/16; C07C 19/08; C07C 31/10

(52) U.S. Cl. CPC C12P 7/04; C07C 31/10; C07C 19/08; C12N 1/16

(57) ABSTRACT

The present invention provides a process for industrial production of chiral-1,1-difluoro-2-propanol. More specifically, a microorganism having the activity to cause asymmetric reduction of 1,1-difluoroacetone or an enzyme having the same activity is allowed to act on 1,1-difluoroacetone, whereby chiral-1,1-difluoro-2-propanol can be produced with high optical purity and in good yield. The process for production of the present invention is easy for industrial implementation.

### TECHNICAL FIELD

The present invention relates to a process for industrial production of chiral-1,1-difluoro-2-propanol which is important as an intermediate for pharmaceuticals and agrochemicals.

### BACKGROUND ART

Chiral-1,1-difluoro-2-propanol is a compound important as an intermediate for various pharmaceuticals and agrochemicals. Until now, Non-patent Document 1 has disclosed a process in which chemical catalysts B-chlorodiisopinocampheylborane (trade name: DIP-Chloride) and B-isopinocampheyl-9-borabicyclo[3.3.1]nonane (registered trademark: R-Alpine-Borane) are allowed to act on 1,1-difluoroacetone to cause asymmetric reduction thereof, thus obtaining chiral-1,1-difluoro-2-propanol. However, the resulting product has low optical purity, i.e., 5% ee (R) and 16% ee (S), which has not reached the optical purity required as an intermediate for pharmaceuticals and agrochemicals. It should be noted that the abbreviation “ee” as used herein refers to enantiomeric excess.

On the other hand, chiral-1,1,1-trifluoro-2-propanol, which is a related compound of chiral-1,1-difluoro-2-propanol, is known to be obtained by processes involving asymmetric reduction of 1,1,1-trifluoroacetone. As to biological processes among such processes, for example, Patent Document 1 discloses a process in which chiral-1,1,1-trifluoro-2-propanol having an optical purity of 99% ee or higher is produced by asymmetric reduction of 1,1,1-trifluoroacetone with alcohol dehydrogenase, Patent Document 2 discloses a process in which (S)-1,1,1-trifluoro-2-propanol of 93% to 99% ee is produced by asymmetric reduction of 1,1,1-trifluoroacetone with commercially available dried baker's yeast, Patent Document 3 discloses a process for production of chiral-1,1,1-trifluoro-2-propanol, which comprises the step of allowing a microorganism functionally expressing an enzyme (e.g., alcohol dehydrogenase, carbonyl reductase) or a transformant thereof or a processed material thereof to act on 1,1,1-trifluoroacetone, Non-patent Document 2 discloses a process in which chiral-1,1,1-trifluoro-2-propanol is obtained by asymmetric reduction of 1,1,1-trifluoroacetone with alcohol dehydrogenase, and Non-patent Document 3 discloses a process in which (S)-1,1,1-trifluoro-2-propanol is obtained with an optical purity of about 80% ee by asymmetric reduction of 1,1,1-trifluoroacetone with dried baker's yeast. In addition, as to chemical processes, for example, Non-patent Document 1 discloses a process in which (S)-1,1,1-trifluoro-2-propanol of 90% ee is obtained by asymmetric reduction of 1,1,1-trifluoroacetone with chemical catalysts. The inventors of the present invention have also disclosed processes with the use of wild-type strains of yeast (Patent Documents 4 and 5).

### PRIOR ART DOCUMENTS

### Patent Documents

Patent Document 1: WO2007/054411

Patent Document 2: WO2007/006650

Patent Document 3: WO2007/142210

Patent Document 4: JP 2011-182787 A

Patent Document 5: JP 2012-5396 A

### Non-Patent Documents

Non-patent Document 1: P. Veeraraghavan Ramachandran et al., Journal of Fluorine Chemistry, vol. 128, pp. 844-850, 2007

Non-patent Document 2: T. C. Rosen et al., Chimica Oggi Suppl., pp. 43-45, 2004

Non-patent Document 3: M. Buccierelli et al., Synthesis, vol. 11, pp. 897-899, 1983

### SUMMARY OF THE INVENTION

### Problem to be Solved by the Invention

As described above, Non-patent Document 1 has disclosed a process involving asymmetric reduction of 1,1-difluoroacetone with chemical catalysts to obtain chiral-1,1-difluoro-2-propanol, although this process resulted in an optical purity as low as 16% ee. In the process of Non-patent Document 1, asymmetric reduction with chemical catalysts was investigated not only for 1,1-difluoro-2-propanol, but also for various other fluorine-containing substrates. In some compounds, their asymmetric reduction reaction was found to proceed with high optical purity (>99% ee), and it has been confirmed that 1,1,1-trifluoro-2-propanol, which is a related compound of 1,1-difluoro-2-propanol intended in the present invention, is obtained with high optical purity (>90% ee). In the case of biological processes, high optical purity (90% ee or higher) has also been confirmed for the related compound chiral-1,1,1-trifluoro-2-propanol in processes with the use of biological enzymes, as found in Patent Document 1, Patent Document 3 and Non-patent Document 2.

In the process of Non-patent Document 1 mentioned above, chemical catalysts were confirmed to ensure high optical purity in 1,1,1-trifluoroacetone. In contrast, when chemical catalysts were allowed to act on 1,1-difluoroacetone, which is a compound having a CF₂H group in place of the CH₃ group in 1,1,1-trifluoroacetone, the stereoselectivity of the catalysts was reduced due to the electronegativity of the substituent and the steric hindrance caused by its hydrogen atom, so that the optical purity was reduced. Such a phenomenon is not limited only to the relationship between 1,1,1-trifluoroacetone and 1,1,-difluoroacetone, and the same phenomenon has also been confirmed in cases where the CH₃ group in CF₃COR (where R is Ph-C, n-Bu-C or n-Hex) is replaced with a CF₂H group (Non-patent Document 1). These CH₃ and CF₂H groups are both fluoromethyl groups which differ only by one in the number of fluorine atoms, but they caused a distinct difference in the stereoselectivity of catalysts. In studies on asymmetric reduction of a ketone adjacent to a difluoromethyl group, chemical catalysts have not been applied successfully, and studies on biological processes were also put off, so that there has been no recent advance in synthesis processes for chiral-1,1-difluoro-2-propanol. Under these circumstances, there has been a demand for the development of, e.g., a catalyst capable of strictly distinguishing the stereostructure of 1,1-difluoroacetone, which is regarded as a pseudosymmetric ketone because the CF₂H and CH₃ groups in its molecule are of almost the same size.

The problem of the present invention is therefore to provide a process for production of chiral-1,1-difluoro-2-propanol with high stereoselectivity.

### Means to Solve the Problem

As a result of extensive and intensive efforts made to solve the problem stated above, the inventors of the present invention have found that when a specific biocatalyst is allowed to act on 1,1-difluoroacetone, asymmetric reduction occurs with high stereoselectivity to thereby obtain chiral-1,1-difluoro-2-propanol of both steric configurations. This finding led to the completion of the present invention.

[Invention 1]

A process for production of chiral-1,1-difluoro-2-propanol represented by the following formula [2], wherein a microorganism having the activity to cause asymmetric reduction of 1,1-difluoroacetone or an enzyme having the same activity is allowed to act on 1,1-difluoroacetone represented by the following formula [1]:

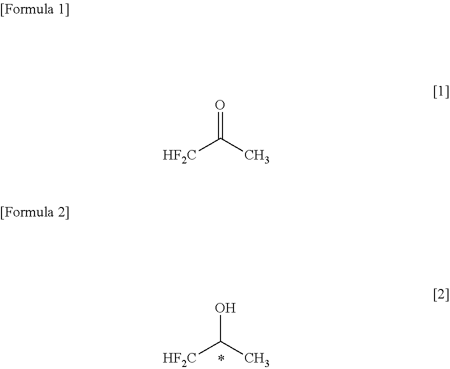

[wherein the asterisk (*) represents an asymmetric atom (the same applies hereinafter in the specification)].

[Invention 2]

The process for production according to Invention 1, wherein the microorganism is at least one selected from the group consisting of Candida guilliermondii, Candida parapsilosis, Candida vini, Candida viswanathii, Cryptococcus laurentii, Cryptococcus curvatus, Debaryomyces maramus, Kluyveromyces marxianus, Ogataea polymorpha, Pichia anomala, Pichia farinosa, Pichia haplophila, Pichia minuta, Rhodotorula muculaginosa, Saccharomyces rouxii, Torulaspora delbrueckii, Wickerhamomyces subpelliculosa, and Zygosaccharomyces rouxii.

[Invention 3]

The process for production according to Invention 2, wherein the microorganism is selected from the microorganisms having Accession Nos. indicated in the table below.

TABLE 1

| | Accession | |
|---|---|---|
| Microorganism | No. | Depository institution |
| Candida guilliermondii | NBRC10279 | National Institute of |
| | | Technology and Evaluation, |
| | | Independent Administrative |
| | | Agency |
| Candida parapsilosis | NBRC0708 | National Institute of |
| | | Technology and Evaluation, |
| | | Independent Administrative |
| | | Agency |
| Candida vini | NBRC1247 | National Institute of |
| | | Technology and Evaluation, |
| | | Independent Administrative |
| | | Agency |
| Candida viswanathii | NBRC10321 | National Institute of |
| | | Technology and Evaluation, |
| | | Independent Administrative |
| | | Agency |
| Cryptococcus laurentii | NBRC0609 | National Institute of |
| | | Technology and Evaluation, |
| | | Independent Administrative |
| | | Agency |
| Cryptococcus curvatus | NBRC1159 | National Institute of |
| | | Technology and Evaluation, |
| | | Independent Administrative |
| | | Agency |
| Debaryomyces maramus | NBRC0668 | National Institute of |
| | | Technology and Evaluation, |
| | | Independent Administrative |
| | | Agency |
| Kluyveromyces marxianus | NBRC10005 | National Institute of |
| | | Technology and Evaluation, |
| | | Independent Administrative |
| | | Agency |
| Ogataea polymorpha | NBRC0799 | National Institute of |
| | | Technology and Evaluation, |
| | | Independent Administrative |
| | | Agency |
| Pichia anomala | NBRC0118 | National Institute of |
| | | Technology and Evaluation, |
| | | Independent Administrative |
| | | Agency |
| Pichia farinosa | NBRC0462 | National Institute of |
| | | Technology and Evaluation, |
| | | Independent Administrative |
| | | Agency |
| Pichia haplophila | NBRC0947 | National Institute of |
| | | Technology and Evaluation, |
| | | Independent Administrative |
| | | Agency |
| Pichia minuta | NBRC0975 | National Institute of |
| | | Technology and Evaluation, |
| | | Independent Administrative |
| | | Agency |
| Rhodotorula muculaginosa | NBRC0001 | National Institute of |
| | | Technology and Evaluation, |
| | | Independent Administrative |
| | | Agency |
| Saccharomyces rouxii | NBRC0493 | National Institute of |
| | | Technology and Evaluation, |
| | | Independent Administrative |
| | | Agency |
| Torulaspora delbrueckii | NBRC0381 | National Institute of |
| | | Technology and Evaluation, |
| | | Independent Administrative |
| | | Agency |
| Wickerhamomyces | NBRC0198 | National Institute of |
| subpelliculosa | | Technology and Evaluation, |
| | | Independent Administrative |
| | | Agency |
| Zygosaccharomyces rouxii | NBRC10671 | National Institute of |
| | | Technology and Evaluation, |
| | | Independent Administrative |
| | | Agency |

[Invention 4]

The process for production according to any one of Inventions 1 to 3, wherein the microorganism is allowed to act as whole microbial cells or as a cell extract thereof.

[Invention 5]

The process for production according to Invention 1, wherein the enzyme is a purified enzyme derived from Ogataea polymorpha, Ogataea parapolymorpha, Pichia anomala, or Pichia minuta.

[Invention 6]

The process for production according to Invention 5, wherein the Ogataea polymorpha is Ogataea polymorpha strain NBRC0799.

[Invention 7]

The process for production according to any one of Inventions 1 to 6, wherein the temperature at which the microorganism or enzyme is allowed to act (i.e., the temperature for reaction) is 5° C. to 60° C.

[Invention 8]

The process for production according to any one of Inventions 1 to 7, wherein the pH at which the microorganism or enzyme is allowed to act (i.e., the pH for reaction) is in the range of 4.0 to 8.0.

[Invention 9]

The process for production according to any one of Inventions 1 to 8, which further comprises a step where the mixture containing 1,1-difluoro-2-propanol and impurities (i.e., the reacted solution) obtained after the microorganism or enzyme is allowed to act (i.e., after completion of the reaction) is distilled to separate the impurities from the mixture and thereby purify 1,1-difluoro-2-propanol.

As described above, attempts have been made to obtain chiral-1,1-difluoro-2-propanol through a process in which chemical catalysts showing high stereoselectivity for 1,1,1-trifluoroacetone are allowed to act on 1,1-difluoroacetone to cause asymmetric reduction thereof in a stereoselective manner, but the optical purity was as low as up to 16% ee. In general, a fluorine atom has a van der Waals radius of 1.47 Å and is close in size to a hydrogen atom (1.20 Å), and it is therefore known that a compound whose hydrogen atom has been replaced with a fluorine atom is also captured by an active site in a biocatalyst or chemical catalyst which recognizes the original compound (this phenomenon is referred to as the mimic effect). However, due to the electronegativity of a fluorine atom and the strength of a C—F bond, etc., such a compound often has different properties (enhanced efficacy, increase or reduced toxicity) from those of the original compound, and is frequently used for pharmaceutical and agrochemical purposes. Likewise, a trifluoromethyl (CF₃) group and a difluoromethyl (CF₂H) group are mutually very similar substituents which differ only by one in the number of fluorine atoms. However, their influence on biocatalysts mostly remains unknown, and hence compounds having these respective substituents should be contacted with biocatalysts to examine their reactivity in actual studies.

Thus, the inventors of the present invention have made extensive and intensive efforts to screen biocatalysts which achieve the object of the present invention from among biocatalysts including whole microbial cells and purified enzymes. As a result, the inventors of the present invention have found a biocatalyst yielding chiral-1,1-difluoro-2-propanol with high optical purity, which has never been achieved by prior art techniques, and this finding led to the completion of the present invention.

During these efforts, the inventors of the present invention have gained very valuable knowledge that two optical isomers of chiral-1,1-difluoro-2-propanol can be prepared separately from each other by changing the biocatalyst to be used.

Moreover, as described in more detail later, the inventors of the present invention have also gained favorable knowledge that the reaction proceeds smoothly upon addition of an organic solvent at a specific concentration.

In the present invention, the concentration of 1,1-difluoroacetone is intended to mean the concentration (w/v) of this acetone in the reaction solution (the concentration of the reduced product is not taken into consideration (i.e., excluded)), but not intended to define the total amount of this acetone added throughout the reaction.

There has been no previous knowledge that a microorganism or enzyme yielding a desired product with high optical purity is found out and is allowed to act on 1,1-difluoroacetone, whereby chiral-1,1-difluoro-2-propanol of both enantiomeric configurations can be produced efficiently with high optical purity (85% ee to 100% ee), as found in the present invention.

### Effects of the Invention

According to the present invention, chiral-1,1-difluoro-2-propanol which is important as an intermediate for pharmaceuticals and agrochemicals can be produced efficiently with high optical purity.

The microorganism or enzyme used in the process for production of the present invention is capable of reducing the carbonyl group of 1,1-difluoroacetone into a hydroxyl group with high optical purity, and further efforts are made to develop a process for asymmetric reduction reaction (e.g., a process for regeneration of the coenzyme NAD(P)H by the action of dehydrogenase without further addition of the coenzyme NAD(P)H from the outside), thereby enabling the provision of chiral-1,1-difluoro-2-propanol with industrially acceptable productivity.

### DESCRIPTION OF EMBODIMENTS

The present invention will be described in more detail below. The scope of the present invention is not limited by the following description, and any embodiments other than those illustrated below may also be carried out with appropriate modifications without departing from the spirit of the invention. It should be noted that this specification incorporates the specification of Japanese Patent Application No. 2015-041696 (filed on Mar. 3, 2015) in its entirety, based on which the present application claims priority.

1,1-Difluoroacetone (represented by the following formula [1]), which is used in the process of the present invention for production of chiral-1,1-difluoro-2-propanol (represented by the following formula [2]) (hereinafter referred to as the process of the present invention), is a known compound and may be prepared as appropriate by those skilled in the art on the basis of prior art techniques, or alternatively, a commercially available product thereof may also be used.

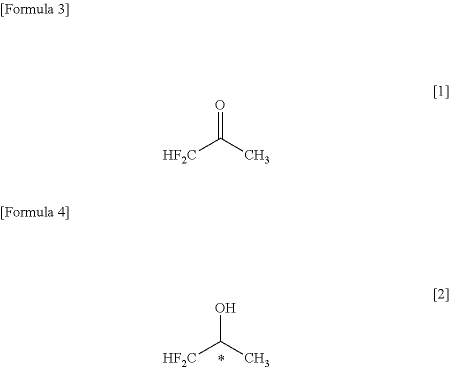

In the process of the present invention, 1,1-difluoroacetone may be of course used directly in compound form, or alternatively, may also be used in admixture with water or an alcohol containing 1 to 4 carbon atoms. This acetone may be directly added to a reaction solution mainly composed of water, but is preferably hydrated before addition because this acetone causes heat generation during hydration with water.

There is no particular limitation on the microorganism which may be used in the process of the present invention, i.e., the microorganism having the activity to reduce 1,1-difluoroacetone into chiral-1,1-difluoro-2-propanol. However, such a microorganism may be exemplified by at least one selected from the group consisting of Candida guilliermondii, Candida parapsilosis, Candida vini, Candida viswanathii, Cryptococcus laurentii, Cryptococcus curvatus, Debaryomyces maramus, Kluyveromyces marxianus, Ogataea polymorpha, Pichia anomala, Pichia farinosa, Pichia haplophila, Pichia minuta, Rhodotorula muculaginosa, Saccharomyces rouxii, Torulaspora delbrueckii, Wickerhamomyces subpelliculosa and Zygosaccharomyces rouxii, preferably exemplified by at least one selected from the group consisting of Candida parapsilosis, Candida vini, Cryptococcus curvatus, Debaryomyces maramus, Kluyveromyces marxianus, Ogataea polymorpha, Pichia anomala, Pichia haplophila, Pichia ininuta, Rhodotorula muculaginosa, Saccharomyces rouxii and Torulaspora delbrueckii, and more preferably exemplified by at least one selected from the group consisting of Cryptococcus curvatus, Kluyveromyces marxianus, Ogataea polymorpha, Pichia anomala, Pichia haplophila, Rhodotorula muculaginosa and Torulaspora delbrueckii.

These microorganisms have been deposited under their respective Accession Nos. indicated in the table below with the National Institute of Technology and Evaluation, Independent Administrative Agency (2-49-10, Nishihara, Shibuya-ku, Tokyo 151-0066, Japan). Some of these microorganisms have also been deposited with other culture collections, and such microorganisms may also be used. It should be noted that these microorganisms are publicly available and can be easily obtained by those skilled in the art.

TABLE 2

| | Accession | |
|---|---|---|
| Microorganism | No. | Depository institution |
| Candida guilliermondii | NBRC10279 | National Institute of |
| | | Technology and Evaluation, |
| | | Independent Administrative |
| | | Agency |
| Candida parapsilosis | NBRC0708 | National Institute of |
| | | Technology and Evaluation, |
| | | Independent Administrative |
| | | Agency |
| Candida vini | NBRC1247 | National Institute of |
| | | Technology and Evaluation, |
| | | Independent Administrative |
| | | Agency |
| Candida viswanathii | NBRC10321 | National Institute of |
| | | Technology and Evaluation, |
| | | Independent Administrative |
| | | Agency |
| Cryptococcus laurentii | NBRC0609 | National Institute of |
| | | Technology and Evaluation, |
| | | Independent Administrative |
| | | Agency |
| Cryptococcus curvatus | NBRC1159 | National Institute of |
| | | Technology and Evaluation, |
| | | Independent Administrative |
| | | Agency |
| Debaryomyces maramus | NBRC0668 | National Institute of |
| | | Technology and Evaluation, |
| | | Independent Administrative |
| | | Agency |
| Kluyveromyces marxianus | NBRC10005 | National Institute of |
| | | Technology and Evaluation, |
| | | Independent Administrative |
| | | Agency |
| Ogataea polymorpha | NBRC0799 | National Institute of |
| | | Technology and Evaluation, |
| | | Independent Administrative |
| | | Agency |
| Pichia anomala | NBRC0118 | National Institute of |
| | | Technology and Evaluation, |
| | | Independent Administrative |
| | | Agency |
| Pichia farinosa | NBRC0462 | National Institute of |
| | | Technology and Evaluation, |
| | | Independent Administrative |
| | | Agency |
| Pichia haplophila | NBRC0947 | National Institute of |
| | | Technology and Evaluation, |
| | | Independent Administrative |
| | | Agency |
| Pichia minuta | NBRC0975 | National Institute of |
| | | Technology and Evaluation, |
| | | Independent Administrative |
| | | Agency |
| Rhodotorula muculaginosa | NBRC0001 | National Institute of |
| | | Technology and Evaluation, |
| | | Independent Administrative |
| | | Agency |
| Saccharomyces rouxii | NBRC0493 | National Institute of |
| | | Technology and Evaluation, |
| | | Independent Administrative |
| | | Agency |
| Torulaspora delbrueckii | NBRC0381 | National Institute of |
| | | Technology and Evaluation, |
| | | Independent Administrative |
| | | Agency |
| Wickerhamomyces | NBRC0198 | National Institute of |
| subpelliculosa | | Technology and Evaluation, |
| | | Independent Administrative |
| | | Agency |
| Zygosaccharomyces rouxii | NBRC10671 | National Institute of |
| | | Technology and Evaluation, |
| | | Independent Administrative |
| | | Agency |

As to the microorganism for use in the process of the present invention, cultured microbial cells may be of course used directly, and it is also possible to use microbial cells homogenized by ultrasonication or with glass beads, microbial cells immobilized with acrylamide or the like, microbial cells treated with an organic compound (e.g., acetone or glutaraldehyde), microbial cells supported on an inorganic carrier (e.g., alumina, silica, zeolite or diatomaceous earth), a cell-free extract or purified enzyme prepared from the microorganism, and a recombinant genetically engineered to carry an enzyme gene cloned from the microorganism.

There is no particular limitation on the enzyme which may be used in the process of the present invention, i.e., the enzyme having the activity to reduce 1,1-difluoroacetone into chiral-1,1-difluoro-2-propanol. However, such an enzyme may be exemplified by alcohol dehydrogenase or carbonyl reductase. Enzymes having the above activity can be selected by screening in which 1,1-difluoroacetone is used as a substrate.

Alcohol dehydrogenase may be exemplified by at least one selected from “Alcohol dehydrogenase, yeast origin” (Oriental Yeast Co., Ltd., Japan), “Alcohol dehydrogenase (ZM-ADH)” (UNITIKA Ltd., Japan), and Chiralscreen® OH E001, E007, E008, E031, E039, E072, E077, E082 and E092 (Daicel Corporation, Japan). Preferred are Chiralscreen® OH E001, E007, E008, E031, E039 and E077, and more preferred are Chiralscreen® OH E001, E031, E039 and E077. Moreover, it is also possible to use a recombinant microorganism genetically engineered to express such an enzyme.

On the other hand, carbonyl reductase may be exemplified by enzymes produced from yeast species of the order Saccharomycetales such as Ogataea polymorpha, Ogataea parapolymorpha, Pichia anomala, Pichia minuta, etc. For purification of these enzymes, techniques commonly used for protein purification can be applied, including ammonium sulfate fractionation, hydrophobic chromatography, ion exchange chromatography, gel filtration chromatography, etc.

For culture of the above microorganisms, it is generally possible to use any nutrient-containing medium (solid medium or liquid medium) for use in microbial culture, although a liquid medium is preferred in the case of reduction reaction of water-soluble 1,1-difluoroacetone. Such a medium comprises a carbon source, as exemplified by sugars (e.g., glucose, sucrose, maltose, lactose, fructose, trehalose, mannose, mannitol, dextrose), alcohols (e.g., methanol, ethanol, propanol, butanol, pentanol, hexanol, glycerol), organic acids (e.g., citric acid, glutamic acid, malic acid), and also comprises a nitrogen source, as exemplified by ammonium salts, peptone, polypeptone, casamino acid, urea, yeast extract, malt extract, corn steep liquor, etc. Further, the medium may be supplemented as appropriate with other medium ingredients such as additional inorganic salts (e.g., potassium dihydrogen phosphate, dipotassium hydrogen phosphate) and vitamins (e.g., inositol, nicotinic acid).

Among the above carbon source, nitrogen source and inorganic salts, the carbon source is preferably added in an amount ensuring the sufficient growth of microorganisms and not inhibiting their growth, and is generally added in an amount of 5 to 80 g, preferably 10 to 40 g, per liter of the medium. Likewise, the nitrogen source is also preferably added in an amount ensuring the sufficient growth of microorganisms and not inhibiting their growth, and is generally added in an amount of 5 to 60 g, preferably 10 to 50 g, per liter of the medium. As to inorganic salts serving as nutrient sources, elements required for microorganism growth should be added, but such an element inhibits the growth at high concentration and is generally added in an amount of 0.001 to 10 g per liter of the medium. It should be noted that these ingredients may be used in any combination depending on the type of microorganism.

The pH in the medium should be adjusted within a range preferred for microorganism growth and is generally set to 4.0 to 10.0, preferably 6.0 to 9.0. The temperature range during culture should be adjusted within a range preferred for microorganism growth and is generally set to 10° C. to 50° C., preferably 20° C. to 40° C. During culture, the medium should be aerated with air preferably at 0.3 to 4 vvm (“vvm” refers to the aeration volume relative to the medium volume per minute and is an abbreviation for volume/volume/minute), more preferably at 0.5 to 2 vvm. In the case of microorganisms with high oxygen demand, the medium may be aerated with air whose oxygen concentration has been increased by using an oxygen generator or the like. Moreover, in the case of test tubes, flasks and other tools which are difficult to set to any aeration volume, the medium volume may be set to 20% or less of the volume of such a tool, and an aeration plug such as a cotton plug or a silicon plug may be provided for the tool. For smooth progress of culture, the medium is preferably stirred. In the case of using a culture vessel, preferably 10% to 100%, more preferably 20% to 90%, of the stirring ability of this vessel is sufficient for this purpose. On the other hand, in the case of small-scale tools such as test tubes and flasks, a shaker may be used for this purpose preferably at 50 to 300 rpm, more preferably at 100 to 250 rpm. The culture time may be set to the time required for microorganism growth to settle down, and culture is continued for 6 to 72 hours, preferably for 12 to 48 hours.

To allow the above microorganism to act on 1,1-difluoroacetone serving as a substrate, a suspension containing the microorganism cultured therein may generally be used directly for the reaction. If the reduction reaction is adversely affected by a component(s) generated during culture, microbial cells (resting microbial cells) obtained by being collected from the cultured solution through centrifugation or other operations may be suspended again to prepare a suspension for use in the reaction. Alternatively, various cell extracts (e.g., a homogenate of the cultured microbial cells, an enzyme prepared from the cultured microbial cells) may also be used for the reaction. On the other hand, when the above enzyme (purified enzyme) is allowed to act on 1,1-difluoroacetone serving as a substrate, the reaction may be conducted in a buffer containing the enzyme dissolved therein. Since this reaction is a reduction reaction, a weakly acidic buffer is preferred for use and examples include sodium phosphate buffer, potassium phosphate buffer, sodium citrate buffer, potassium citrate buffer, sodium acetate buffer and potassium acetate buffer.

For efficient progress of the above microorganism-mediated reaction, there is a necessity to increase the density of these microbial cells in a suspension. However, at too high a density, the reaction is inhibited in some cases due to autolytic enzyme production and end metabolite accumulation, etc. Thus, the reaction is generally conducted at 10⁶ to 10¹² cfu/ml (“cfu” refers to the number of colonies formed on an agar medium and is an abbreviation for colony forming units), preferably at 10⁷ to 10¹¹ cfu/ml, and more preferably at 10⁸ to 10¹⁰ cfu/ml. On the other hand, for efficient progress of the above enzyme-mediated reaction, there is a necessity to increase the concentration of the enzyme in a buffer. However, the excess use of the enzyme is not cost effective, and the enzyme is used preferably in the range of 0.01 g/L to 20 g/L, more preferably in the range of 0.1 g/L to 10 g/L.

When 1,1-difluoroacetone is added to such a suspension or buffer, the concentration of this acetone is preferably maintained at a concentration which allows smooth progress of the reduction reaction and does not adversely affect the activity of the microorganism or enzyme. If the concentration of this acetone is higher than 5% (w/v), the microorganism may be killed or the enzyme may be denatured; and hence the concentration is maintained lower than this numerical value, i.e., generally 0.01% to 10% (w/v), preferably 0.05% to 6% (w/v). As to the volumetric basis on which the concentration of this acetone is calculated, for example, the culture solution volume dispensed into test tubes before autoclaving may be taken into account in Example 1 described later, while the total suspension volume of the cultured microorganism may be taken into account in Example 3 described later.

The temperature at which the above microorganism or enzyme is allowed to act on 1,1-difluoroacetone serving as a substrate (i.e., the temperature for reaction) should be maintained within a range preferred for the microorganism or enzyme, and is generally 5° C. to 60° C., preferably 15° C. to 50° C., and more preferably 15° C. to 38° C. Likewise, the pH at which the microorganism or enzyme is allowed to act (i.e., the pH for reaction) should also be maintained within a preferred range, and is generally 4.0 to 8.0, preferably 5.5 to 8.0, and more preferably 5.5 to 7.0.

If the microorganism suspension or the enzyme-containing buffer is in a standing state, the reaction efficiency will be reduced, so that the reaction is conducted while stirring the reaction solution. Moreover, the reaction may be conducted under non-aeration conditions, but may optionally be conducted under aeration conditions when required. In this case, if the aeration volume is too high, 1,1-difluoroacetone and chiral-1,1-difluoro-2-propanol may be released as gases into the external environment; and hence the aeration volume is preferably 0.3 vvm or less, and more preferably 0.1 vvm or less. The reaction time may be determined depending on the progress of desired product production and is generally 6 to 312 hours.

In the present invention, the coenzyme NAD(P)H (serving as a hydrogen donor) for use in the reduction reaction is regenerated from the coenzyme NAD(P) by the action of dehydrogenase inherent to the microorganism or dehydrogenase integrated into E. coli. Thus, an additional substrate serving as a hydrogen source is preferably presented in the suspension during the reduction reaction, and sugars and/or alcohols may also be used for this purpose. As to the coenzyme NAD(P)H, a commercially available product thereof may further be added during the reduction reaction, but such a commercially available product is very expensive and therefore not cost effective. As intended in the present invention, the coenzyme NAD(P)H is regenerated by the action of dehydrogenase without further addition of the coenzyme NAD(P)H from the outside, whereby the number of reductions per microbial cell is increased and a desired product can be produced with high cost effectiveness and high productivity.

The process of the present invention is directed to converting 1,1-difluoroacetone to chiral-1,1-difluoro-2-propanol by industrial production process. The process of the present invention is capable of producing chiral-1,1-difluoro-2-propanol in a large amount by preferred reaction conditions.

It should be noted that in the process of the present invention, an optically active alcohol, i.e., chiral-1,1-difluoro-2-propanol can be obtained with an optical purity of 85% ee or higher, particularly preferably 98% ee or higher, which is also practically acceptable.

In the case of using whole microbial cells, many oxidoreductases coexist in the microbial cells, so that the optical purity is reduced as a whole. However, the optical purity can be improved by using a desired enzyme in purified form.

To collect the thus produced chiral-1,1-difluoro-2-propanol from the reacted solution (i.e., the mixture containing impurities and others obtained after completion of the reaction), isolation techniques commonly used in organic synthesis may be used. After completion of the reaction, a crude product can be obtained by standard post-handling operations such as distillation or extraction with an organic solvent. In particular, when the reacted solution or the washed filtrate optionally treated to remove the microbial cells is directly provided for distillation, chiral-1,1-difluoro-2-propanol can be collected in a simple manner and in high yield. The resulting crude product may optionally be subjected to purification operations such as dehydration, activated charcoal, fractional distillation, column chromatography and so on.

### EXAMPLES

Some illustrative examples will be given below, but the present invention is not limited to the following examples.

### Example 1

### Examination (Screening) Results for the Reactivity of Microorganisms to 1,1-Difluoroacetone

A liquid medium composed of distilled water (1000 ml), polypeptone (10 g), yeast extract (5 g) and sodium chloride (10 g) was prepared and dispensed in 5 ml volumes into test tubes (ϕ1.8 cm×18 cm), and then inoculated with the respective microorganisms indicated in Table A below, followed by culture at 28° C. at 160 spm for 24 hours. After completion of the culture, 1,1-difluoroacetone was added at 1% wt/v to conduct reduction reaction at 28° C. at 160 rpm for 24 hours. The conversion rate after the reaction was measured by ¹⁹F-NMR (internal standard). For measurement of the optical purity, the reaction solution was mixed with ethyl acetate to extract 1,1-difluoro-2-propanol into the organic layer, which was then analyzed by gas chromatography using a chiral column as described later. The conversion rate and optical purity were measured for each microorganism used, and the results obtained are shown in Table A below. (The same procedures were used hereinafter to measure the conversion rate and the optical purity.)

TABLE 3
Table A

| | | Con- | | Steric |
|---|---|---|---|---|
| | Accession | version | Optical | config- |
| Microorganism | No. | rate | purity | uration |
| Candida guilliermondii | NBRC10279 | 81.8% | 78.3% ee | S |
| Candida parapsilosis | NBRC0708 | 67.9% | 81.8% ee | S |
| Candida vini | NBRC1247 | 78.4% | 82.3% ee | S |
| Candida viswanathii | NBRC10321 | 81.6% | 79.5% ee | S |
| Cryptococcus laurentii | NBRC0609 | 19.6% | 76.9% ee | S |
| Cryptococcus curvatus | NBRC1159 | 43.3% | 90.3% ee | S |
| Debaryomyces maramus | NBRC0668 | 9.7% | 81.8% ee | S |
| Kluyveromyces | NBRC10005 | 53.6% | 90.7% ee | S |
| marxianus | | | | |
| Ogataea polymorpha | NBRC0799 | 63.5% | 86.8% ee | S |
| Pichia anomala | NBRC0118 | 14.0% | 89.8% ee | S |
| Pichia farinosa | NBRC0462 | 81.5% | 75.0% ee | S |
| Pichia haplophila | NBRC0947 | 10.6% | 83.8% ee | S |
| Pichia minuta | NBRC0975 | 93.3% | 80.5% ee | S |
| Rhodotorula | NBRC0001 | 100.0% | 90.1% ee | S |
| muculaginosa | | | | |
| Saccharomyces rouxii | NBRC0493 | 62.2% | 86.5% ee | S |
| Torulaspora | NBRC0381 | 16.1% | 85.6% ee | S |
| delbrueckii | | | | |
| Wickerhamomyces | NBRC0198 | 32.7% | 79.5% ee | S |
| subpelliculosa | | | | |
| Zygosaccharomyces | NBRC10671 | 68.4% | 72.7% ee | S |
| rouxii | | | | |

[Analysis Conditions for Gas Chromatography Using a Chiral Column]

For use as a test sample, 1,1-difluoroacetone extracted into ethyl acetate was derived into its acetoxy form by being reacted with 1.2 equivalents of acetic anhydride and 1.2 equivalents of pyridine. For gas chromatography, a Cyclosil-B column (0.25 mm×30 m×0.25 μm; Agilent Technologies) was used, and analysis conditions were set as follows: carrier gas: nitrogen; pressure: 100 kPa; column temperature: 60° C. to 90° C. (1° C./min) up to 150° C. (10° C./min); and vaporizer/detector (FID) temperature: 230° C. The peaks obtained under these analysis conditions were measured for their area to calculate the optical purity. The respective enantiomers of 1,1-difluoro-2-propanol were found to have a retention time of 4.6 minutes (S-configuration) and 5.3 minutes (R-configuration). Their steric configuration was determined by the new Mosher's method using (-)-Mosher's acid chloride (¹H-NMR chemical shift: [S-MTPA ester] CF₂H 6.06, H 5.42, CH₃ 1.44, [R-MTPA ester] CF₂H 6.18, H 5.44, CH₃ 1.36).

### Example 2

### Production of (S)-1,1-difluoro-2-propanol by Means of Whole Microbial Cells

For use as a pre-culture medium, a liquid medium composed of distilled water (1000 ml), polypeptone (10 g), yeast extract (5 g) and sodium chloride (10 g) was prepared and dispensed in 5 ml volumes into test tubes (ϕ1.8 cm×18 cm), and then autoclaved at 121° C. for 15 minutes. Into this liquid medium, Ogataea polymorpha strain NBRC0799 was aseptically inoculated with a platinum loop and cultured at 28° C. at 160 rpm for 16 hours to obtain a pre-culture solution at 1.4×10⁷ cfu/ml. For use as a main culture medium, a liquid medium composed of distilled water (500 ml), glucose (16.25 g), yeast extract (12.5 g), polypeptone (7.5 g), potassium dihydrogen phosphate (1.2 g), dipotassium hydrogen phosphate (0.625 g) and a defoaming agent (FC2901, Asahi Kasei Corporation, Japan; 0.2 g) was prepared and charged into a culture vessel of 1 L volume (model BME01, ABLE Corporation, Japan), and then autoclaved at 121° C. for 15 minutes. Into this culture vessel, 5 ml of the pre-culture solution was aseptically inoculated and cultured at 28° C. under 1 vvm aeration while stirring at 700 rpm for 18 hours to prepare a suspension at 1.7×10⁹ cfu/ml (28 g/L calculated as a wet cell weight). The pH during culture was adjusted to pH 6.5 with 20% aqueous sodium bicarbonate and 42.5% aqueous phosphoric acid. After completion of the culture, the aeration volume was changed to 0 vvm and the culture solution was mixed with 1,1-difluoroacetone at 1% wt/v and glucose at 6.25% wt/v to conduct reduction reaction at 28° C. for 43 hours. After the reaction, the conversion rate was found to be 100% and the optical purity was found to be 93.4% ee (S). The reacted culture solution was directly distilled to collect an aqueous solution of (S)-1,1-difluoro-2-propanol (9.8 g). This aqueous solution was dehydrated by addition of calcium hydroxide (anhydrous) to thereby obtain (S)-1,1-difluoro-2-propanol (4.1 g) at a moisture content of 2.1%.

### Example 3

### Production of (S)-1,1-difluoro-2-propanol by Means of a Cell-Free Extract

The microbial cell suspension of Ogataea polymorpha strain NBRC0799 cultured in Example 2 was transferred to centrifugal tubes of 500 ml volume and centrifuged at 18,000×g for 30 minutes to collect the microbial cells. The collected wet microbial cells were diluted with 15 ml of 0.2 M phosphate buffer (pH 7.0) to prepare a suspension. The cells in the suspension were homogenized with a bead-based cell homogenizer (Bead Beater, BioSpec) and, after removal of the glass beads, were centrifuged at 20,000×g for 30 minutes to prepare a cell-free extract. This cell-free extract (1 ml) was mixed with 1,1-difluoroacetone at 1% wt/v and 2 M glucose (250 μL) to conduct reduction reaction at 28° C. for 24 hours. After the reaction, the conversion rate was found to be 100% and the optical purity was found to be 95.6% ee (S).

### Example 4

### Purification of Carbonyl Reductase from Ogataea polymorpha Strain NBRC0799

After test tubes (ϕ1.4 cm×18 cm) each containing 5 ml of a liquid medium (pH 6.5) composed of glucose (10 g/L), peptone (5 g/L), yeast extract (3 g/L), malt extract (3 g/L), potassium dihydrogen phosphate (3 g/L) and dipotassium hydrogen phosphate (2.0 g/L) were autoclaved at 121° C. for 15 minutes, Ogataea polymorpha strain NBRC0799 was aseptically inoculated with a platinum loop and cultured at 30° C. at 300 rpm for 24 hours to prepare a pre-pre-culture solution at 3.84×10¹⁰ cfu/ml.

A 500 ml Erlenmeyer flask containing 200 ml of the above liquid medium was autoclaved at 121° C. for 15 minutes, and 2 ml of the pre-culture solution was aseptically added thereto, followed by culture at 30° C. while stirring at 180 rpm for 24 hours to prepare a pre-culture solution at 3.8×10¹⁰ cfu/ml.

The same medium as shown above was dispensed in 1000 ml volumes into Sakaguchi flasks of 2 L volume and then autoclaved at 121° C. for 15 minutes. To these flasks, the pre-culture solution was aseptically added in 10 ml volumes and cultured at 30° C. at 96 rpm for 24 hours. The culture solutions were transferred to centrifugal tubes of 500 ml volume and centrifuged at 3000×g for 8 minutes to collect the microbial cells.

[Procedures for Enzyme Activity Measurement]

For enzyme activity measurement, NADH was added at a final concentration of 0.1 mM to 168 mM sodium phosphate buffer (pH 6.0) containing an enzyme or a microorganism, and 1,1-difluoroacetone was added at a final concentration of 50 mM to this reaction solution to initiate the reaction (reaction solution volume: 1 mL). The reaction was conducted at 30° C., and decreases in NADH were monitored with a spectrophotometer (V-630BIO, JASCO Corporation, Japan) as changes in absorbance at 340 nm. It should be noted that the enzyme activity was determined assuming that the amount of enzyme required to catalyze the oxidation of 1 μmol NADH per minute was defined as 1 U (unit).

[Preparation of a Cell-Free Extract]

The collected microbial cells were diluted with 5 volumes of 10 mM sodium phosphate buffer (pH 7.0) to prepare a suspension. The microbial cells were homogenized with a bead-based cell homogenizer (Multi-beads shocker, Yasui Kikai Corporation, Japan) and centrifuged at 20,000×g for 10 minutes, and the resulting supernatant was used as a cell-free extract.

[Ammonium Sulfate Fractionation]

The cell-free extract was mixed with ammonium sulfate at 30% saturated ammonium sulfate concentration and stirred on ice for 3 hours. After centrifugation at 20,000×g for 30 minutes, the supernatant was used as a 30% saturated ammonium sulfate fraction for the subsequent purification step.

[Hydrophobic Chromatography—Phenyl-Toyopearl]

The above 30% saturated ammonium sulfate supernatant fraction was applied to a 60 ml Phenyl-Toyopearl column (TOYOPEARL® Phenyl-650M, Tosoh Corporation, Japan) which had been equilibrated with a 30% saturated ammonium sulfate solution. The column was washed with 300 ml of 10 mM phosphate buffer (pH 7.0) containing 300 mM ammonium sulfate and then eluted with 10 mM sodium phosphate buffer. The resulting active fractions were pooled and used as a Phenyl-Toyopearl active fraction for the subsequent purification step.

[Ion Exchange Chromatography—Q-sepharose]

The above Phenyl-Toyopearl active fraction was applied to a 30 ml Q-sepharose column (Q-sepharose Fast Flow, G E Healthcare UK Ltd.) which had been equilibrated with 10 mM sodium phosphate buffer (pH 7.0). Subsequently, the column was washed with 150 ml of 10 mM sodium phosphate buffer (pH 7.0) and then eluted with a linear gradient of 0 to 150 mM sodium chloride. The resulting active fractions were used as a Q-sepharose active fraction for the subsequent purification step.

[Hydroxyapatite Column Chromatography—Hydroxyapatite]

The Q-sepharose active fraction was applied to a 10 mL hydroxyapatite column (Nacalai Tesque, Inc., Japan) which had been equilibrated with 10 mM phosphate buffer (pH 7.0). The column was washed with 100 ml of 10 mM sodium phosphate buffer (pH 7.0) and then eluted with a linear gradient formed from 10 mM and 300 mM sodium phosphate buffers (pH 7.0). Fractions showing activity were pooled and concentrated with a centrifugal filter unit (Amicon Ultra-15, 10 kDa, Merck Millipore) to isolate an enzyme.

The purified enzyme was eventually found to have a specific activity of 29.8 U/mg.

### Example 5

### Confirmation of the Reactivity of Ogataea polymorpha Strain NBRC0799-Derived Carbonyl Reductase to 1,1-difluoroacetone

To 168 mM sodium phosphate buffer (pH 6.0) containing the enzyme purified from Ogataea polymorpha strain NBRC0799, NADH was added at a final concentration of 0.1 mM, and the substrates indicated in Table B below were each added at a final concentration of 50 mM to this reaction solution to initiate the reaction (reaction solution volume: I mL). The reaction was conducted at 30° C., and decreases in NADH were measured with a spectrophotometer (V-630BIO, JASCO Corporation, Japan) as changes in absorbance at 340 nm.

Relative activity refers to the ratio of enzyme activity (conversion speed) for each substrate in comparison with the enzyme activity when using acetone as a substrate, which is defined to be 100%. When 1,1-difluoroacetone or 1,1,1-trifluoroacetone is provided as a substrate, the conversion speed per se of the enzyme is reduced (many hours are required to complete the reaction). In the case of acetone, its two methyl groups are equal, so that acetone will be captured by a recognition site in the enzyme without distinction between these two methyl groups. However, in the case of 1,1-difluoroacetone and 1,1,1-trifluoroacetone, their capture by an active site in the enzyme would require recognition between a fluoromethyl group and a methyl group, so that the speed of their capture becomes slower. On the other hand, in the case of 1,3-difluoroacetone, the fluorine atoms introduced thereinto would alter electronegativity and so on, when compared to acetone.

TABLE 4
Table B

| | Relative |
|---|---|
| | Activity |
| Substrate | (%) |
| (a) Reduction reaction | |
| Acetone | 100 |
| Fluoroacetone | 211 |
| 1,1-Difluoroacetone | 58 |
| 1,3-Difluoroacetone | 52 |
| 1,1,1-Trifluoroacetone | 50 |
| 4,4,4-Trifluoro-2-butanone | 228 |
| 2-Butanone | 132 |
| Formaldehyde | 16 |
| Acetaldehyde | 330 |
| Propionaldehyde | 141 |
| Butyraldehyde | 193 |

### Example 6

### Examination (Screening) Results for the Reactivity of Commercially Available Alcohol Dehydrogenases to 1,1-difluoroacetone

To 1 ml of 200 mM potassium phosphate buffer (pH 6.5) containing 206 mM sodium formate, 222 mM glucose, 5 mM NAD⁺ (NAD⁺: nicotinamide adenine dinucleotide, oxidized form; the same applies hereinafter) and 5 mM NADP⁺(NADP⁺: nicotinamide adenine dinucleotide phosphate, oxidized form; the same applies hereinafter), 1,1-difluoroacetone was added to account for 1% by weight, and the alcohol dehydrogenases (Chiralscreen® OH, Daicel Corporation, Japan) indicated in the “Enzyme name” column in Table C below were each added in an amount of 5 mg, followed by reaction at 25° C. for 2 days while stirring with a magnetic stirrer. After the reaction, the conversion rate and optical purity were measured and are shown in Table C below.

TABLE 5
Table C

| Enzyme | Reaction | Conversion | | Steric |
|---|---|---|---|---|
| name | time | rate | Optical purity | configuration |
| E001 | 22 h | 100% | 96.68% ee | S |
| E007 | 22 h | 100% | 76.20% ee | S |
| E008 | 22 h | 100% | 83.04% ee | S |
| E031 | 19 h | 100% | 98.40% ee | S |
| E039 | 19 h | 100% | 96.62% ee | R |
| E072 | 22 h | 13% | 83.94% ee | S |
| E077 | 22 h | 18% | 96.82% ee | S |
| E082 | 22 h | 84% | 60.66% ee | S |
| E092 | 24 h | 100% | 61.36% ee | S |

### Comparative Example 1

The same procedures as shown in Example 5 were repeated to evaluate the alcohol dehydrogenases (Chiralscreen® OH, Daicel Corporation, Japan) indicated in the “Enzyme name” column in Table D below for their reactivity to 1,1-difluoroacetone. The results obtained are shown in Table D below.

TABLE 6
Table D

| Enzyme | Reaction | Conversion | | Steric |
|---|---|---|---|---|
| name | time | rate | Optical purity | configuration |
| E004 | 22 h | 0% | Not measured | Not determined |
| E005 | 22 h | 0% | Not measured | Not determined |
| E019 | 22 h | 0% | Not measured | Not determined |
| E021 | 19 h | 0% | Not measured | Not determined |
| E048 | 19 h | 0% | Not measured | Not determined |
| E051 | 19 h | 0% | Not measured | Not determined |
| E057 | 19 h | 0% | Not measured | Not determined |
| E073 | 22 h | 0% | Not measured | Not determined |
| E078 | 22 h | 0% | Not measured | Not determined |
| E079 | 22 h | 0% | Not measured | Not determined |
| E080 | 22 h | 0% | Not measured | Not determined |
| E085 | 45 h | 0% | Not measured | Not determined |
| E086 | 24 h | 0% | Not measured | Not determined |
| E087 | 24 h | 0% | Not measured | Not determined |
| E119 | 24 h | 0% | Not measured | Not determined |
| E128 | 24 h | 0% | Not measured | Not determined |
| E146 | 24 h | 0% | Not measured | Not determined |

### Example 7

### Production of (R)-1,1-difluoro-2-propanol by Means of Recombinant E. coli Genetically Engineered to Express Alcohol Dehydrogenase

For use as a pre-culture medium, a liquid medium composed of distilled water (1000 ml), polypeptone (10 g), yeast extract (5 g) and sodium chloride (10 g) was prepared and dispensed in 5 ml volumes into test tubes (ϕ1.6 cm×15 cm), and then autoclaved at 121° C. for 15 minutes. Into this liquid medium, recombinant E. coli genetically engineered to overexpress Chiralscreen® OH E031 alcohol dehydrogenase (Daicel Corporation, Japan) was aseptically inoculated with a platinum loop and cultured overnight at 30° C. at 160 rpm to obtain a pre-culture solution whose optical density at a wavelength of 600 nm (OD600) was 7.7.

For use as a main culture medium, a liquid medium containing yeast extract, sodium glutamate, glucose, lactose, inorganic salts and a defoaming agent in distilled water (2000 ml) was prepared and charged into a culture vessel of 5 L volume (model MDN 5L(S), B. E. Marubishi Co., Ltd., Japan), and then autoclaved at 121° C. for 30 minutes. Into this culture vessel, 5 ml of the pre-culture solution was aseptically inoculated and cultured at 30° C. under 0.5 vvm aeration while stirring for 40 hours to prepare a suspension whose optical density (OD600) was 24. The pH during culture was adjusted to around pH 7.0 with 28% aqueous ammonia and 50% aqueous phosphoric acid. After completion of the culture, the aeration volume was changed to 0 vvm and the culture solution was mixed with 1,1-difluoroacetone at 3.6% wt/v (72 g) to conduct reduction reaction at 20° C. at pH 6.5 for 24 hours while regenerating the coenzyme. After the reaction, the conversion rate was found to be 100% and the optical purity was found to be 96.9% ee (R).

The reacted culture solution was directly distilled to collect an aqueous solution of (R)-1,1-difluoro-2-propanol (102 g). This aqueous solution was dehydrated by addition of calcium hydroxide (anhydrous) to thereby obtain (R)-1,1-difluoro-2-propanol (70 g) at a moisture content of 1.2%. This (R)-1,1-difluoro-2-propanol (70 g) was subjected to fractional distillation using a ϕ2 cm×30 cm rectifying column filled with Helipack packing No. 1 (TO-TOKU Engineering Corporation, Japan) to collect fractions at steam temperatures of 87° C. to 88° C. The collected fractions were subjected to gas chromatography using a Cyclosil-B column (0.25 mm×30 m×0.25 μm; Agilent Technologies) [carrier gas: nitrogen; pressure: 100 kPa; column temperature: 60° C. to 90° C. (1° C./min) up to 150° C. (10° C./min); and vaporizer/detector (FID) temperature: 230° C.]. The resulting peaks were used to calculate the area of 1,1-difluoro-2-propanol relative to the total area. As a result, the area of 1,1-difluoro-2-propanol was found to account for 99.0% of the total area.

### INDUSTRIAL APPLICABILITY

According to the present invention, chiral-1,1-difluoro-2-propanol which is important as an intermediate for pharmaceuticals and agrochemicals can be produced efficiently with high optical purity.

The microorganism or enzyme used in the process for production of the present invention is capable of reducing the carbonyl group of 1,1-difluoroacetone into a hydroxyl group with high optical purity, and further efforts are made to develop a process for asymmetric reduction reaction (e.g., a process for regeneration of the coenzyme NAD(P)H by the action of dehydrogenase without further addition of the coenzyme NAD(P)H from the outside), thereby enabling the provision of chiral-1,1-difluoro-2-propanol with industrially acceptable productivity.

1. A process for production of chiral-1,1-difluoro-2-propanol represented by formula [2], wherein a microorganism having the activity to cause asymmetric reduction of 1,1-difluoroacetone or an enzyme having the same activity is allowed to act on 1,1-difluoroacetone represented by formula [1]: 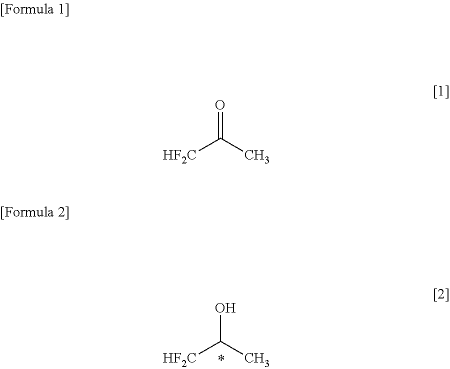
[wherein the asterisk (*) represents an asymmetric atom].

2. The process for production according to claim 1, wherein the microorganism is at least one selected from the group consisting of Candida guilliermondii, Candida parapsilosis, Candida vini, Candida viswanathii, Cryptococcus laurentii, Cryptococcus curvatus, Debaryomyces maramus, Kluyveromyces marxianus, Ogataea polymorpha, Pichia anomala, Pichia farinosa, Pichia haplophila, Pichia minuta, Rhodotorula muculaginosa, Saccharomyces rouxii, Torulaspora delbrueckii, Wickerhamomyces subpelliculosa, and Zygosaccharomyces rouxii.

3. The process for production according to claim 2, wherein the microorganism is selected from the microorganisms having Accession Nos. indicated in the table below.
TABLE 1

| | Accession | |
|---|---|---|
| Microorganism | No. | Depository institution |
| Candida guilliermondii | NBRC10279 | National Institute of |
| | | Technology and Evaluation, |
| | | Independent Administrative |
| | | Agency |
| Candida parapsilosis | NBRC0708 | National Institute of |
| | | Technology and Evaluation, |
| | | Independent Administrative |
| | | Agency |
| Candida vini | NBRC1247 | National Institute of |
| | | Technology and Evaluation, |
| | | Independent Administrative |
| | | Agency |
| Candida viswanathii | NBRC10321 | National Institute of |
| | | Technology and Evaluation, |
| | | Independent Administrative |
| | | Agency |
| Cryptococcus laurentii | NBRC0609 | National Institute of |
| | | Technology and Evaluation, |
| | | Independent Administrative |
| | | Agency |
| Cryptococcus curvatus | NBRC1159 | National Institute of |
| | | Technology and Evaluation, |
| | | Independent Administrative |
| | | Agency |
| Debaryomyces maramus | NBRC0668 | National Institute of |
| | | Technology and Evaluation, |
| | | Independent Administrative |
| | | Agency |
| Kluyveromyces marxianus | NBRC10005 | National Institute of |
| | | Technology and Evaluation, |
| | | Independent Administrative |
| | | Agency |
| Ogataea polymorpha | NBRC0799 | National Institute of |
| | | Technology and Evaluation, |
| | | Independent Administrative |
| | | Agency |
| Pichia anomala | NBRC0118 | National Institute of |
| | | Technology and Evaluation, |
| | | Independent Administrative |
| | | Agency |
| Pichia farinosa | NBRC0462 | National Institute of |
| | | Technology and Evaluation, |
| | | Independent Administrative |
| | | Agency |
| Pichia haplophila | NBRC0947 | National Institute of |
| | | Technology and Evaluation, |
| | | Independent Administrative |
| | | Agency |
| Pichia minuta | NBRC0975 | National Institute of |
| | | Technology and Evaluation, |
| | | Independent Administrative |
| | | Agency |
| Rhodotorula muculaginosa | NBRC0001 | National Institute of |
| | | Technology and Evaluation, |
| | | Independent Administrative |
| | | Agency |
| Saccharomyces rouxii | NBRC0493 | National Institute of |
| | | Technology and Evaluation, |
| | | Independent Administrative |
| | | Agency |
| Torulaspora delbrueckii | NBRC0381 | National Institute of |
| | | Technology and Evaluation, |
| | | Independent Administrative |
| | | Agency |
| Wickerhamomyces | NBRC0198 | National Institute of |
| subpelliculosa | | Technology and Evaluation, |
| | | Independent Administrative |
| | | Agency |
| Zygosaccharomyces rouxii | NBRC10671 | National Institute of |
| | | Technology and Evaluation, |
| | | Independent Administrative |
| | | Agency |

4. The process for production according to any one of claims 1 to 3, wherein the microorganism is allowed to act as whole microbial cells or as a cell extract thereof.

5. The process for production according to claim 1, wherein the enzyme is a purified enzyme derived from Ogataea polymorpha, Ogataea parapolymorpha, Pichia anomala, or Pichia minuta.

6. The process for production according to claim 5, wherein the Ogataea polymorpha is Ogataea polymorpha strain NBRC0799.

7. The process for production according to claim 1, wherein the temperature at which the microorganism or enzyme is allowed to act is 5° C. to 60° C.

8. The process for production according to claim 1, wherein the pH at which the microorganism or enzyme is allowed to act is in the range of 4.0 to 8.0.

9. The process for production according to claim 1, which further comprises a step where the mixture containing 1,1-difluoro-2-propanol and impurities obtained after the microorganism or enzyme is allowed to act is distilled to separate the impurities from the mixture and thereby purify 1,1-difluoro-2-propanol.
